(19) United States
(12) Patent Application Publication
(10) Pub. No.: US 2015/0366576 A1
(43) Pub. Date: Dec. 24, 2015

(54) ULTRASOUND-GUIDED MINIMALLY INVASIVE PLANTAR FASCIA RELEASE

(71) Applicant: THAYER INTELLECTUAL PROPERTY, INC., Lafayette, CA (US)

(72) Inventors: Edward Liou, Pleasanton, CA (US); David Michael Schummers, San Francisco, CA (US); Jeffrey D. Smith, Clayton, CA (US); Bruce M. McCormack, San Francisco, CA (US)

(21) Appl. No.: 14/749,494

(22) Filed: Jun. 24, 2015

Related U.S. Application Data
(60) Provisional application No. 62/016,563, filed on Jun. 24, 2014.

(51) Int. Cl. A61B 17/32

(52) U.S. Cl. CPC A61B 17/320016; A61B 2017/320052; A61B 17/3403

(57) ABSTRACT

A method for performing a plantar fasciotomy on a foot may involve advancing an elongate plantar fasciotomy device through an entry point and an exit point on the foot, cutting the plantar fascia, using the plantar fasciotomy device, and observing the advancing step and/or the cutting step, via an ultrasound imaging device.

### CROSS REFERENCE TO RELATED APPLICATION

This application claims the benefit of priority to U.S. Application No. 62/016,563 entitled Ultrasound-Guided Minimally Invasive Plantar Fascia Release, filed Jun. 24, 2014, which is hereby incorporated by reference.

### FIELD OF THE INVENTION

The present disclosure relates to medical devices and methods. More specifically, it relates to methods and devices for treating plantar fasciitis.

### BACKGROUND

Plantar fasciitis is the most common cause of plantar heel pain, affecting up to 10% of the adult population in the United States. Approximately 10% to 20% of patients with recalcitrant heel pain will require operative treatment. FIG. 1 illustrates the anatomy and procedure target area. A foot 100 is shown. The plantar fascia 104 extends from the heel bone 102 to the proximal phalanges of the toes. An area of pain 106 that is typically associated with plantar fasciitis is indicated adjacent to the heel bone 102.

The surgical procedure is known as a plantar fasciotomy or plantar fascia release. It involves the partial transection of the plantar fascia to release tension and relieve inflammation. Plantar fascia releases have traditionally been performed using an open surgical approach, but significant issues have been identified, including post-operative complications due to a non-healing wound, the patient being unable to walk on the foot for several weeks, and the potential for injuring or damaging nerves that traverse the fascia. Patients who are offered open surgery often decline it because of the long recovery time.

Current practices favor minimally invasive techniques, which yield decreased postoperative pain and edema and quicker return to activity. It is believed the number of surgical cases will increase if an effective, minimally invasive option becomes widely available.

Although endoscopic plantar fasciotomy has been in use since the early 1990's the risk of calcaneal nerve injury, incomplete release, long operating room set-up and steep learning curve for the surgeon have limited its adoption and use.

Therefore, it would be advantageous to have improved methods for performing plantar fasciotomy procedures minimally invasively, addressing the current issues associated with the endoscopic method. Ideally, these methods would allow for minimally invasive access with minimal system setup, simple operation, clear visibility of the anatomy, and effective fascia release. At least some of these objectives will be met by the embodiments described herein.

### BRIEF SUMMARY

The embodiments described herein provide methods for accessing the plantar fascia via a percutaneous approach and performing plantar fasciotomy under ultrasound guidance. The embodiments described below generally include an ultrasound imaging system, providing images of internal anatomy to guide the procedure. The embodiments described below generally include a surgical tool that will allow for insertion into or near the plantar fascia via a minimally invasive access method. The surgical device described herein generally includes a cutting feature that can perform the fasciotomy without endangering nearby tissues and nerves.

The methods described herein may include and/or may be performed using a minimally invasive plantar fasciotomy device, such as the device illustrated in FIG. 3. One example of such a plantar fasciotomy device is the Manos plantar fasciotomy device, from Thayer Intellectual Property, Inc. (www.thayerhealth.com).

A method for performing a plantar fasciotomy on a foot is disclosed. The method includes advancing an elongate plantar fasciotomy device through an entry point and an exit point on the foot, cutting the plantar fascia, using the plantar fasciotomy device, and observing at least one of the advancing step or the cutting step, via an ultrasound imaging device. The method may further include at least one of the following steps: administering local anesthesia, mapping the entry point and the exit point, using anatomical landmarks; using the ultrasound imaging device to confirm locations of the entry point and exit point; creating the entry point by making a small incision on the skin of the foot; advancing an access cannula through the entry point; and piercing the plantar fascia with the access cannula. In some embodiments, the method may further include using the ultrasound imaging device to verify proper placement of the access cannula. In some embodiments, the method may further include using the ultrasound imaging device to verify proper placement of the cannula in the plantar fascia or advancing the cutting device through the access cannula and cutting the plantar fascia with the cutting device, or withdrawing the access cannula prior to cutting the plantar fascia, or using the ultrasound imaging device to monitor and/or measure the progress of cutting of the plantar fascia and confirm fascia release. The method may be minimally invasive.

A method for performing a plantar fasciotomy on a foot is disclosed. In one aspect, the method includes mapping an entry point and an exit point on a foot; introducing an access cannula through the entry point; directing the access cannula at the exit point using an ultrasound imaging device; introducing a cutting device through the access cannula; and transecting the plantar fascia using the cutting device. The method may further include verifying the transection of the plantar fascia using the ultrasound imaging device. In some aspects, the method may further include administering local anesthesia. The method may further include mapping the entry and exit points using the ultrasound imaging device. Mapping the entry and exit points comprises confirming that the entry and exit points are less than one centimeter from a distal portion of a calcaneal tuberosity. In some aspects, the method may include piercing the plantar fascia with the access cannula and directing the access cannula at the exit point. The access cannula does not exit the plantar fascia during the piercing in some aspects.

A method for performing a plantar fasciotomy on a foot is disclosed. In some aspects, the method includes introducing an access cannula through an entry point into a plantar fascia; verifying, by ultrasound imaging, a location of the access cannula in the plantar fascia; advancing a cutting device through the access cannula, the cutting device having a cutting surface and a wire; and transecting the plantar fascia with the cutting surface. In some aspects, the method may include monitoring the plantar fascia with an ultrasound imaging device during the transecting of the plantar fascia. The wire may exit the foot at an exit point. Advancing the cutting device may include withdrawing the access cannula from the plantar fascia to expose the cutting surface. In some aspects, the access cannula is inserted into the plantar fascia to a depth of between approximately two and approximately three centimeters.

These and other aspects and embodiments will be described in further detail below, in reference to the attached drawing figures.

### BRIEF DESCRIPTION OF DRAWINGS

FIG. 1 is an illustration of the foot anatomy and procedure target area.

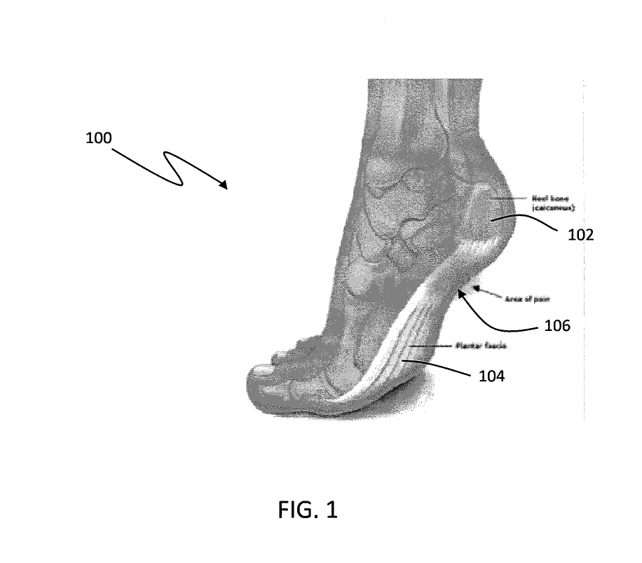

FIG. 2 is a flowchart depicting a method for ultrasound guided plantar fascia release.

FIG. 3 is an illustration of a plantar fasciotomy device, according to one embodiment.

FIGS. 4A and 4B are medial and lateral/bottom views of a foot, illustrating entry and exit points for a cutting device, according to one embodiment.

FIG. 5 is a representative view of an ultrasound image showing the internal anatomy around the plantar fascia.

FIG. 6 is an illustration of the cutting tool position in the plantar fascia, prior to fascia transection, according to one embodiment.

FIG. 7 is a representative view of an ultrasound image showing the confirmation of plantar fascia release.

### DETAILED DESCRIPTION

Embodiments of the present invention recognize that traditional methods of plantar fasciotomy suffer from several drawbacks. For example, open surgical methods may result in post-operative complications due to a non-healing wound, the patient being unable to walk on the foot for several weeks, and/or injuring or damaging nerves that traverse the fascia. Embodiments described in this disclosure provide a minimally invasive method for performing a plantar fasciotomy with improved recovery time and decreased risk of nerve damage.

According to one embodiment, a method for treating plantar fasciitis may include the steps described below. Those skilled in the art will appreciate that the steps described herein are described by way of example, one or more steps may be deleted or completed in a different order and should not be interpreted as limiting the scope of the attached claims.

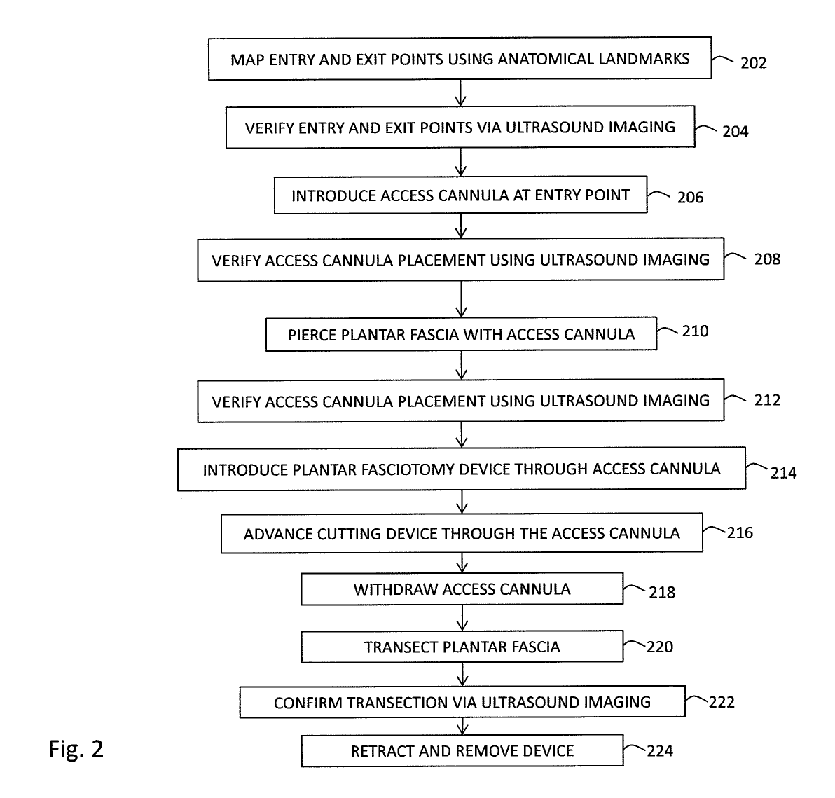

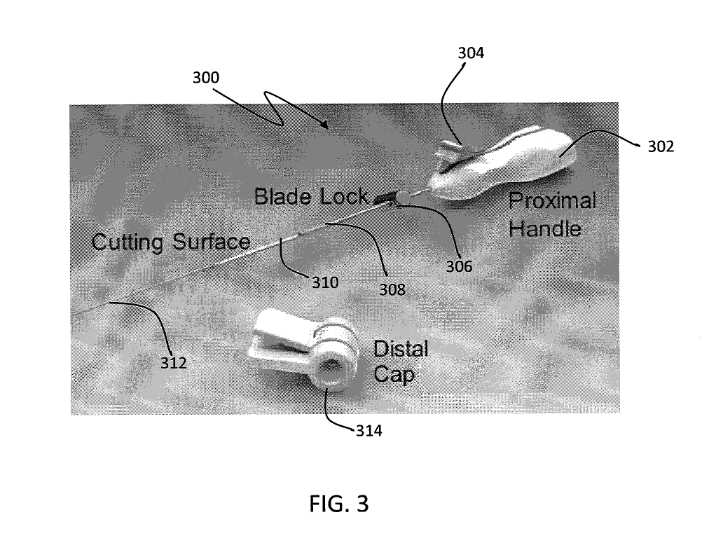

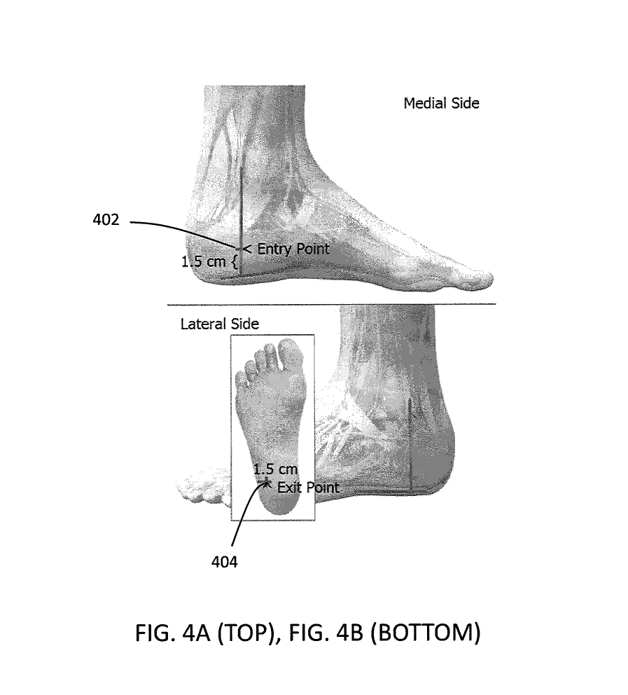

FIG. 2 is a flowchart depicting a method for ultrasound guided plantar fascia release and FIG. 3 is an illustration of a plantar fasciotomy device that may be used in embodiments of the method. Initially, a physician, anesthesiologist, or other qualified person may administer anesthesia to the patient. The anesthesia used may be any appropriate anesthesia and may include local anesthesia, general anesthesia, sedatives, or a combination thereof In step 202, the surgeon or other clinician maps entry and exit points using anatomical landmarks. The entry point is located on the posterior edge of the medial malleolus (inner ankle bone). Using a marker, a line is dropped down toward the plantar surface on the sole of the neutrally positioned foot. The surgeon finds the plantar surface and marks the skin change on the medial side of the foot. In one embodiment, the entry point is marked 1.5 cm proximal from the plantar surface on the line from the medial malleolus. FIG. 4A illustrates the entry point location. To map the exit point, the posterior edge of the lateral malleolus (outer ankle bone) is located. Using a marker, a line is dropped down toward the plantar surface and the lateral border of plantar surface is identified, and the skin change on the lateral border is marked. In one embodiment, the exit point is marked on the plantar surface 1.5 cm medial from the lateral border. Those skilled in the art will appreciate that the entry and exit points should be aligned with one another. FIG. 4B illustrates the exit point location.

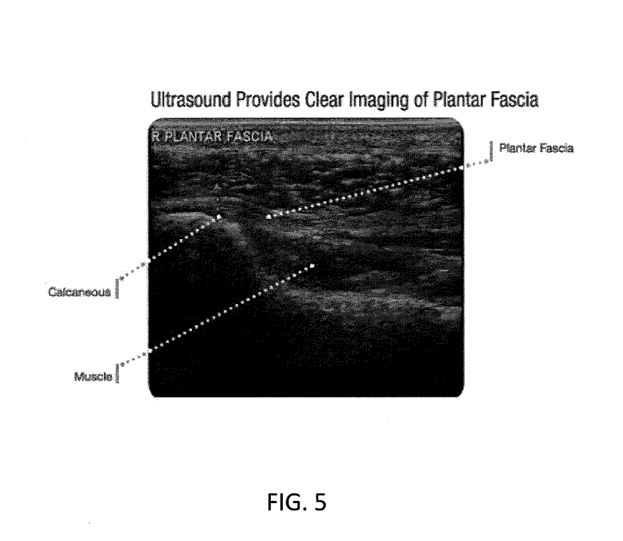

In step 204, ultrasound imaging is used to confirm that the entry and exit points are as close to the calcaneal tuberosity as possible, i.e., less than approximately 1 cm distal to the calcaneal tuberosity. FIG. 5 illustrates a representative view of the ultrasound image.

In step 206, the access cannula is introduced at the entry point. Using a surgical blade, a skin nick is created through the skin at the entry point, for example, a small stab incision. In this embodiment, an access cannula is inserted parallel to the plantar surface to a depth of approximately 2 cm to approximately 3 cm. In step 208, the cannula placement is confirmed using ultrasound on the plantar surface (the sole of the foot). Confirming the placement may include a step of ensuring that the cannula is deep to the plantar fascia (e.g., between approximately 2 cm and approximately 3 cm) and not superficial.

In step 210, the plantar fascia is pierced with the access cannula. In various embodiments, the access cannula is withdrawn slightly, so that the access cannula can pivot at the entry point and be directed toward the exit point. The access cannula is then angled toward the exit point and inserted into the plantar fascia towards the exit point. The cannula pierces the plantar fascia but does not exit the plantar surface. In step 212, the position of the access cannula in the plantar fascia is confirmed with ultrasound.

In step 214, a plantar fasciotomy device is introduced to the target site through the access cannula. In some embodiments, a plantar fasciotomy device such as that shown in FIG. 3 may be used. An example device is described in U.S. Pat. No. 8,348,966, which is hereby incorporated by reference.

Referring now to FIG. 3, an example plantar fasciotomy device 300 is shown. The plantar fasciotomy device 300 may include a handle 302, a thumb slide 304, a blade lock 306, an elongate portion 308, a cutting surface 310, a needle tip 312, and a distal cap 314. The thumb slide 304 may be coupled to the handle 302 and operable to advance and/or retract the elongate portion 308, cutting surface 310, and/or the needle tip 312. In various embodiments, the plantar fasciotomy device 300 may be adjusted to a retracted position by retracting the thumb slide 304 such that the cutting surface 310 and the needle point 312 are housed within the elongate portion 308 and/or the handle 302. The plantar fasciotomy device 300 may be adjusted to an extended position by advancing the thumb slide 304 such that the cutting surface 310 and the needle tip 312 are extended out of the elongate portion 308 and exposed to surrounding tissue (e.g., the plantar fascia). The blade lock 306 may be fixed to the elongate portion 308 to hold the cutting surface in place relative to the handle 302 while in the extended position. The distal cap 314 may be fixed to the needle tip 312 to prevent the needle tip 312 from being withdrawn through plantar fascia during the plantar fasciotomy procedure.

Referring again to step 214 in FIG. 2, the plantar fasciotomy device (e.g., plantar fasciotomy device 300) is introduced through the access cannula in the retracted position (e.g., the needle tip 312 and cutting surface 310 hidden from view). The plantar fasciotomy device is advanced until it tents against the plantar surface at or near the previously marked exit point.

In step 216, the cutting surface is advanced through the access cannula. For example, the surgeon may advance the cutting surface 310 by advancing the thumb slide 304 on the handle 302 of the plantar fasciotomy device 300. This deploys the cutting surface 310 through the plantar fascia and also pushes the needle tip 312 through the exit point. The distal cap 314 may be attached to the needle tip 312 to keep needle tip 312 from being withdrawn through the exit point prematurely. In various embodiments, the access cannula may remain between the cutting surface 310 and the plantar fascia.

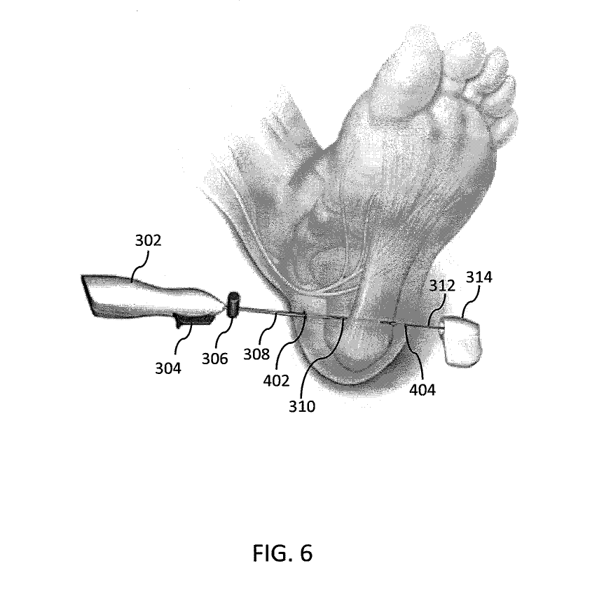

In step 218, the surgeon withdraws the access cannula. In various embodiments, the surgeon slides the access cannula toward the handle of the plantar fasciotomy device to allow the cutting surface to contact the fascia. FIG. 6 illustrates the plantar fasciotomy device in position after the access canella has been withdrawn. As shown in FIG. 6, the elongate portion 308 extends through the entry point 402. The cutting surface 310 contacts the plantar fascia. The needle tip 312 extends through the exit point 404. The distal cap 314 is fixed to the end of the needle tip 312 to prevent the needle tip from being withdrawn through the exit point 404 during the plantar fasciotomy procedure.

In step 220, the plantar fascia is transected. In some embodiments, the plantar fasciotomy device 300 is gripped towards the top of the handle 302. A downward force is applied on the handle 302. In response to the downward force, the plantar fasciotomy device 300 bows upwards, bringing the cutting surface 310 into direct contact with the plantar fascia. Pressure is applied to the sole of the foot to increase the contact between the cutting surface 310 and the plantar fascia. In some embodiments, the repeated application and release of pressure to the plantar fasciotomy device and/or the sole of the foot results in transection of the plantar fascia. In some embodiments, the surgeon may apply a force to the handle 302 to draw and push the cutting surface in a sawing motion transverse to the plantar fascia to facilitate the transection. In various embodiments, the surgeon cuts towards the medial aspect.

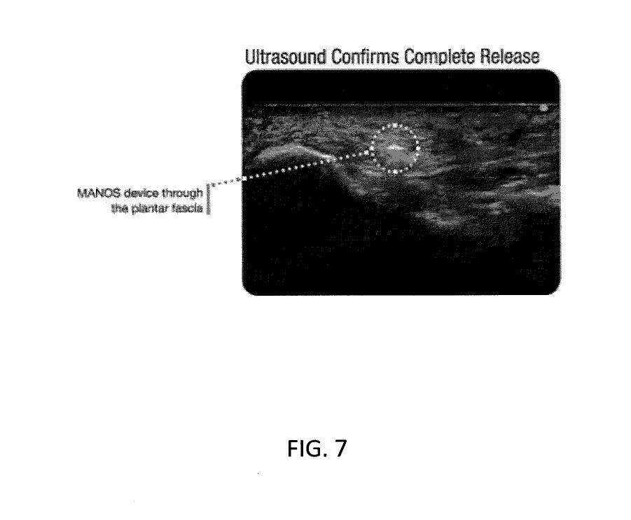

In step 222, the surgeon may monitor and/or measure the progress of transection of the plantar fascia using ultrasound imaging. In some embodiments, the surgeon may periodically monitor the progress of the transection with ultrasound imaging. In other embodiments, the surgeon may monitor the progress with ultrasound imaging throughout the duration of the plantar fasciotomy procedure. FIG. 7 illustrates a representative view of the ultrasound image. Once the release of the plantar fascia is confirmed via ultrasound imaging in step 222, the plantar fasciotomy device is retracted and removed in step 324. For example, the surgeon may remove the distal cap 314 from the needle tip 312, and withdraw the cutting surface 310 and needle tip 312 by moving the thumb slide 304 to the retracted position.

All relative and directional references (including: upper, lower, upward, downward, left, right, leftward, rightward, top, bottom, side, above, below, front, middle, back, vertical, horizontal, and so forth) are given by way of example to aid the reader's understanding of the particular embodiments described herein. They should not be read to be requirements or limitations, particularly as to the position, orientation, or use unless specifically set forth in the claims. Connection references (e.g., attached, coupled, connected, joined, and the like) are to be construed broadly and may include intermediate members between a connection of elements and relative movement between elements. As such, connection references do not necessarily infer that two elements are directly connected and in fixed relation to each other, unless specifically set forth in the claims.

Although the invention has been disclosed in the context of certain embodiments and examples, the present invention extends beyond the specifically disclosed embodiments to other alternative embodiments and/or uses of the invention and obvious modifications and equivalents thereof. Thus, it is intended that the scope of the present invention herein disclosed should not be limited by the particular disclosed embodiments described above.

What is claimed is:

1. A method for performing a plantar fasciotomy on a foot, the method comprising:
advancing an elongate plantar fasciotomy device through an entry point and an exit point on the foot;
cutting the plantar fascia, using the plantar fasciotomy device; and
observing at least one of the advancing step or the cutting step, via an ultrasound imaging device.

2. The method of claim 1, further comprising at least one of the following steps:
administering local anesthesia;
mapping the entry point and the exit point, using anatomical landmarks;
using the ultrasound imaging device to confirm locations of the entry point and exit point;
creating the entry point by making a small incision on the skin of the foot;
advancing an access cannula through the entry point;
piercing the plantar fascia with the access cannula

3. The method of claim 2, further comprising
using the ultrasound imaging device to verify proper placement of the access cannula.

4. The method of claim 2, further comprising
using the ultrasound imaging device to verify proper placement of the cannula in the plantar fascia.

5. The method of claim 1, further comprising
advancing the cutting device through the access cannula and cutting the plantar fascia with the cutting device.

6. The method of claim 5, further comprising
withdrawing the access cannula prior to cutting the plantar fascia.

7. The method of claim 1, further comprising
using the ultrasound imaging device to monitor and/or measure the progress of cutting of the plantar fascia and confirm fascia release.

8. The method of claim 2, wherein the method is minimally invasive.

9. A method for performing a plantar fasciotomy on a foot, the method comprising:
mapping an entry point and an exit point on a foot;
introducing an access cannula through the entry point;
directing the access cannula at the exit point using an ultrasound imaging device;
introducing a cutting device through the access cannula; and
transecting the plantar fascia using the cutting device.

10. The method of claim 9, further comprising:
verifying the transection of the plantar fascia using the ultrasound imaging device.

11. The method of claim 9, further comprising:
administering local anesthesia.

12. The method of claim 9, further comprising:
mapping the entry and exit points using the ultrasound imaging device.

13. The method of claim 12, wherein mapping the entry and exit points comprises confirming that the entry and exit points are less than one centimeter from a distal portion of a calcaneal tuberosity.

14. The method of claim 9, further comprising:
piercing the plantar fascia with the access cannula and directing the access cannula at the exit point.

15. The method of claim 14, wherein the access cannula does not exit the plantar fascia during the piercing.

16. A method for performing a plantar fasciotomy on a foot, the method comprising:
introducing an access cannula through an entry point into a plantar fascia;
verifying, by ultrasound imaging, a location of the access cannula in the plantar fascia;
advancing a cutting device through the access cannula, the cutting device having a cutting surface and a wire.
transecting the plantar fascia with the cutting surface.

17. The method of claim 16, further comprising:
monitoring the plantar fascia with an ultrasound imaging device during the transecting of the plantar fascia.

18. The method of claim 16, wherein the wire exits the foot at an exit point.

19. The method of claim 16, wherein advancing the cutting device comprises withdrawing the access cannula from the plantar fascia to expose the cutting surface.

20. The method of claim 16, wherein the access cannula is inserted into the plantar fascia to a depth of between approximately two and approximately three centimeters.
